(19) United States
(12) Patent Application Publication
(10) Pub. No.: US 2012/0188512 A1
(43) Pub. Date: Jul. 26, 2012

(54) Lens Assembly for Improving Phoropter Performance

(75) Inventors: Dwight Duston, Laguna Niguel, CA (US); Anthony Van Heugten, Sarasota, FL (US)

(73) Assignee: e-Vision Smart Optics, Inc., Roanoke, VA (US)

(21) Appl. No.: 13/395,513

(22) PCT Filed: Apr. 16, 2010
(86) PCT No.: PCT/US2010/031353

§ 371 Date: Apr. 11, 2012

Related U.S. Application Data
(60) Provisional application No. 61/243,184, filed on Sep. 17, 2009.
(60) Provisional application No. 61/254,230, filed on Oct. 23, 2009.

(51) Int. Cl. A61B 3/028

(52) U.S. Cl. 351/233; 351/246

(57) ABSTRACT

Aspects of the present invention provide systems, methods, and apparatuses for providing coarse vision correction tuning capability, fine vision correction tuning capability, and/or high-order aberration correction capability. An optical device or lens assembly of the present invention can include one or more conventional lenses, one or more fluid or liquid lenses, one or more electro-active lenses, or any combination thereof. The optical device or lens assembly can be mechanically, adhesively, or magnetically coupled to a phoropter or can be built into the phoropter as an integrated add [symbol] on lens assembly. Electro-active lenses within the lens assembly of the present invention can provide a range of optical powers including—positive, neutral (plano), and negative optical powers—that can be discretely or continuously tuned or adjusted. A wired or wireless remote unit can be used to control the lens assembly of the present invention.

### CROSS-REFERENCE TO RELATED APPLICATIONS

This application claims priority from and incorporates by reference in their entirety the following provisional applications:

U.S. Appl. No. 61/243,184, filed on Sep. 17, 2009; and

U.S. Appl. No. 61/254,230, filed on Oct. 23, 2009.

### BACKGROUND OF THE INVENTION

1. Field of the Invention

The present invention generally relates to electro-active optical systems. More specifically, the present invention provides electro-active optical systems providing fine vision correction tuning and high-order aberration correction to improve vision acuity examinations.

2. Background Art

Conventional phoropters or refractors are diagnostic instruments used to measure the refractive error of a patient. Currently, eye examinations are performed entirely by the eye care professional. Typically, the eye care professional adjusts the various optical lens elements of the phoropter that are placed in the optical path of the patient's eye as the patient views an eye chart. The patient's participation in the examination is minimal and generally does not extend beyond providing subjective feedback to the eye care professional on whether an adjusted optical element improves or degrades visual acuity. Thus, while the patient provides some input on the acuity to the eye care professional, adjustments to the optical elements of the phoropter are left entirely to the discretion of the eye care professional.

Recently, eye care professionals have been encouraged to involve the patient more in the examination process. It is believed that more patient involvement can result in improved “buy-in” or confidence of the patient in the final determined prescription. In turn, this would lead to greater patient satisfaction and fewer examination “re-do's”.

Accordingly, what are needed are improved examination methods and optical devices and systems to facilitate improved examinations. Further, what are needed are systems and methods that provide the patient with more control over the final stages of the eye examination, as the final vision prescription is conclusively determined. Additionally, what are needed are optical devices and systems to enable more precise vision prescriptions to be determined such that a patient can experience fine vision correction (e.g., spherical and/or cylindrical correction) and high-order aberration correction.

### BRIEF DESCRIPTION OF THE DRAWINGS/FIGURES

FIG. 1 illustrates an optical device of the present invention.

FIG. 2 provides a flowchart that illustrates operational steps for providing fine vision correction tuning during a vision acuity examination in accordance with an aspect of the present invention.

FIG. 3 provides a flowchart that illustrates operational steps for providing a high-order aberration correction in accordance with an aspect of the present invention.

### DETAILED DESCRIPTION OF THE INVENTION

Aspects of the present invention provide systems, methods, and apparatuses for providing coarse vision correction tuning capability, fine vision correction tuning capability, and/or high-order aberration correction capability. Coarse and fine vision correction can comprise spherical and/or cylindrical correction. An optical device or lens assembly of the present invention can include one or more conventional lenses, one or more fluidic (or fluid) or liquid lenses, one or more electro-active lenses, or any combination thereof. The optical device or lens assembly can be mechanically, adhesively, or magnetically coupled to a phoropter or can be built into the phoropter as an integrated add-on lens assembly. Electro-active lenses within the lens assembly of the present invention can provide a range of optical powers—including positive, neutral (plano), and negative optical powers—that can be discretely or continuously tuned or adjusted. A wired or wireless remote unit can be used to control the lens assembly of the present invention.

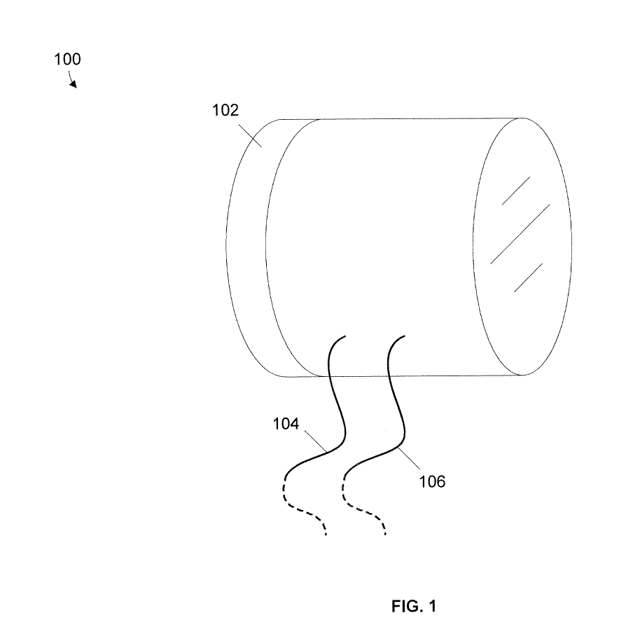

FIG. 1 illustrates an optical device 100. The optical device 100 can be used to improve or optimize the performance of a conventional phoropter. The optical device 100 can include one or more electro-active lenses, one or more fluid or liquid lenses, one or more conventional lenses, or any combination thereof. The optical device 100 can be used to provide coarse vision correction tuning and/or fine vision correction tuning (e.g., fine or coarse cylindrical and/or spherical correction). The optical device can also be used to provide correction for high-order aberrations.

The optical device 100 can be attached or coupled to a conventional phoropter. More specifically, the optical device 100 can be positioned within a first optical path or a first eye-well of a conventional phoropter (with a second optical device, for example, positioned in a second optical path or second eye-well of the conventional phoropter). Alternatively, the optical device 100 (or at least a portion of its constituent optical, power and control elements) can be built into a conventional phoropter as part of an integrated optimizer lens assembly. Under either scenario, the one or more lenses of the optical device 100 can be positioned to be in optical communication with the lenses of the phoropter. Accordingly, the lenses of the phoropter can provide a coarse vision correction for a patient and the one or more lenses of the optical device can provide an additive fine vision correction (that together can form a total or final patient prescription when optically combined). Fine tuning capability can be on the order of a fraction of a Diopter while coarse tuning capability can be on the order of several Diopters.

The optical device 100 can comprise a mechanism 102 for attaching to a conventional phoropter. The optical device 100 can either be attached to the front or the back of a conventional phoropter (i.e., either the side closer to a patient or the side further from the patient). The mechanism 102 can be a mechanical means for attaching—such as, but not limited to, screws and fasteners—and/or can include the use of adhesives. The mechanism 102 can also be a means for magnetically coupling the optical device 100 to a conventional phoropter.

The optical device 100 can further comprise a power connection 104. The optical device 100 can be powered by either a remote AC or DC power source or can include an internal (e.g., rechargeable) power source. The power connection 104 can be electrically coupled to a power source of a phoropter to which it is attached or can be coupled to a separate power source.

The optical device 100 can also comprise a control connection 106. The control connection 106 can couple the optical device 100 to a remote control unit (not depicted in FIG. 1). The remote control unit can be used by either the patient or the individual conducting the patient's eye exam (e.g., the eye exam administrator). As an alternative to a wired control connection, the optical device 100 can include a wireless communication unit that can interact with one or more remote wireless control units.

The optical device 100, as depicted in FIG. 1, is shown to be an enclosed cylindrical unit but is not so limited. That is, the lens assembly of the present invention can form any attachable or add-on device having the above-mentioned power, optical and communication components to provide coarse or fine vision correction tuning and/or high-order aberration correction. More generally, the optical device 100 can be any lens assembly (either as an add-on component or integrated into a conventional phoropter) having one or more lenses in optical communication (e.g., in series) to enable the optical power provided by each lens to be additive.

According to an aspect of the present invention, the optical device 100 can include one or more electro-active lenses that can be used to provide fine vision correction tuning (e.g., to form a fine tuning lens assembly). As used herein, an electro-active lens refers to a lens that has a variable optical power that can be adjusted electrically, either by a driving voltage, electro-magnetic field, or other electrical means and includes the electro-active lenses described in U.S. Pat. No. 5,712,721, U.S. Pat. No. 6,517,203, U.S. patent application Ser. No. 12/408,973, filed Mar. 23, 2009, U.S. Pat. No. 7,264,354, U.S. patent application Ser. No. 12/135,587, filed Jun. 9, 2008, and U.S. patent application Ser. 12/410,889, filed Mar. 25, 2009, each of which are hereby incorporated by reference in their entirety. Each electro-active lens within the optical device 100 can provide an adjustable positive, neutral and/or negative optical power.

As an example, the optical device 100 can include a single electro-active lens. The single electro-active lens can have three discrete optical power settings such as −0.25 D, 0.0 D (i.e., plano), and +0.25 D by providing discrete optical steps of 0.25 D. Alternatively, the single electro-active lens can have five discrete optical power settings such as +0.5 D, +0.25 D, 0.0 D, −0.25 D and −0.5 D. As a further alternative, the single electro-active lens of the optical device 100 can provide a different set of five optical power settings such as +0.25 D, +0.125 D, 0.0 D, −0.125 D, and −0.25 D by providing discrete optical steps of 0.125 D.

As an additional example, the single electro-active lens of the optical device 100 can be a continuously tunable electro-active lens. The continuously tunable electro-active lens can provide analog or nearly-analog tunability over some limited range of optical powers.

According to an aspect of the present invention, the astigmatic axis of a cylindrical lens of the optical device 100 or phoropter to which the optical device 100 is coupled (or incorporate within) can be adjusted by manual, automatic or motorized rotation of the lens or by electro-optic variation of the cylindrical lens axis.

As mentioned above, the optical device 100 can be controlled by a remote wired or wireless unit. The remote control unit can be used the patient and/or the individual conducting the eye exam. Further, the optical device 100 can include at least two remote control units—one for use by the patient and one for use by the eye exam administrator.

An individual operating the remote control unit can use the remote control unit to activate and deactivate the electro-active lenses of the optical device 100. By interacting with the controls of the remote control unit, a user of the remote control unit can toggle between discrete or continuous optical power settings or ranges provided by the optical device 100.

The remote control unit used in association with the optical device 100 can include a power source (e.g., batteries or a rechargeable power unit) or can include a wired connection for coupling to a power source. The remote control unit associated with the optical device 100 can include a display for indicating a selected optical power setting of the optical device 100. Alternatively, or in addition thereto, the remote control unit can indicate the power setting using a connected display device—for example, a display unit coupled wirelessly or with a wired connection to the remote control unit or optical device 100.

For an integrated optimizer lens assembly built into a conventional phoropter, a remote control unit can also be used to adjust the optical power of the optimizer lens assembly. Alternatively, additional controls on the phoropter can be used by the patient or individual conducting the eye exam to adjust the power of the electro-optic lens assembly. The optical power provided by the integrated lens assembly can then be electronically displayed on the phoropter itself or on the remote control unit.

The optical device 100 (or an integrated lens assembly version of the optical device 100) can be used to enhance a conventional visual acuity examination. For example, during a refractive examination, the administrator of the vision exam can use the conventional lenses of a phoropter to provide a coarse correction of the patient's vision (to determine a first correction component—e.g., comprising coarse cylindrical and/or spherical correction components). The patient or administrator can then activate the optical device 100 to provide a fine tuning correction of the patient's vision by adjusting the optical power provided by the optical device 100 (to determine a second or supplemental correction component—e.g., comprising fine cylindrical and/or spherical correction components). The fine tuning correction provided by the optical device 100 can be experienced by the patient in conjunction with the coarse correction provided by the conventional phoropter. By changing the optical power provided by the optical device 100, a final and more precise vision correction can be determined.

The optical device 100 can also include one more electro-active lenses that can be used to correct for high-order aberrations including, but not limited to, spherical aberration, trefoil, coma, multi-axis or irregular astigmatism. High-order aberrations of a patient can be determined, for example, by measuring the aberrations with a wavefront sensor. Such a wavefront sensor can be a stand-alone device or can be integrated into a phoropter. After measuring the aberrations, the optical device 100 can be operated to use the one or more electro-active lenses to correct for these high-order aberrations. As a result, the patient can be provided or can experience visual acuity that is enhanced when compared to what is achievable using a conventional standard examination only.

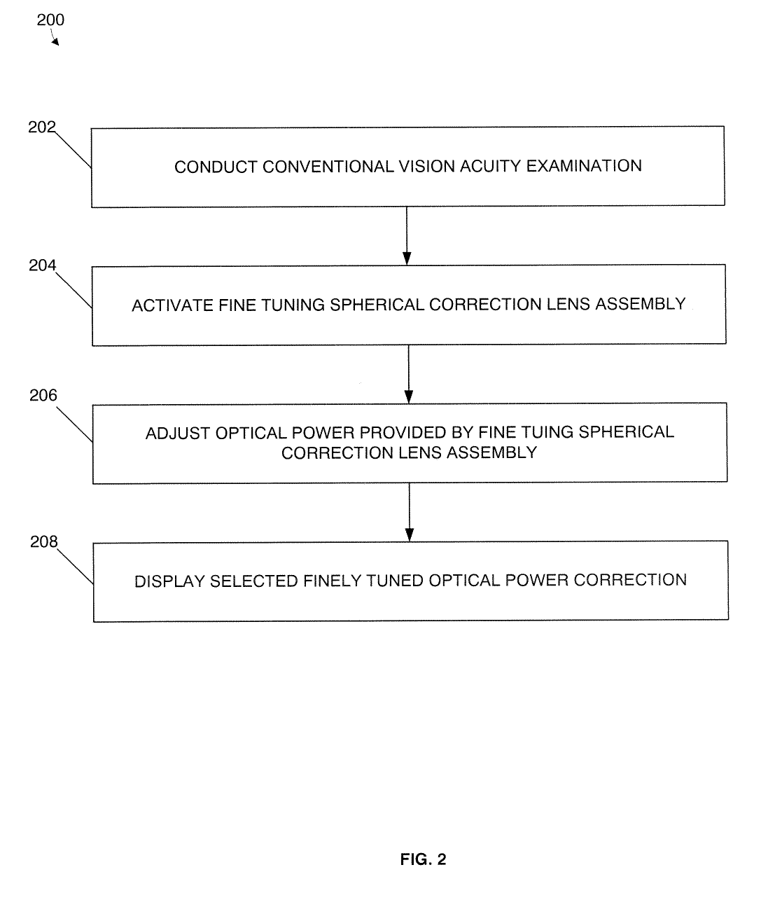

FIG. 2 provides a flowchart 200 that illustrates operational steps for providing fine vision correction tuning during a vision acuity examination in accordance with an aspect of the present invention. The invention is not limited to this operational description. Rather, it will be apparent to persons skilled in the relevant art(s) from the teachings herein that other operational control flows are within the scope and spirit of the present invention. In the following discussion, the steps in FIG. 2 are described.

At step 202, a typical or conventional vision acuity examination can be conducted. The vision acuity examination can be conducted on a patient by an eye exam administrator. The vision acuity exam can be conducted using a conventional phoropter. At the end of step 202, a coarse vision refractive correction of the patient can be determined.

At step 204, a lens assembly providing fine vision correction tuning capability of the present invention can be activated. The lens assembly can be an add-on lens assembly (such as the optical device 100 described above) or can be a lens assembly incorporated into the design and fabrication of the conventional phoropter. The lens assembly can include one or more electro-active lenses, one or more fluid or liquid lenses, one or more conventional lenses, or any combination thereof. As an example, the lens assembly of the present invention can include a single electro-active lens providing a range of positive and negative optical powers. The lens assembly can be placed within the optical path of the patient's eye such that the patient experiences the combined optical powers of the conventional phoropter (coarse correction) and fine tuning lens assembly (fine correction).

At step 206, an optical power provided by the fine tuning lens assembly can be adjusted. The optical power provided by the fine tuning lens assembly can be adjusted by the patient and/or the administrator of the eye examination. The optical power provided by the fine tuning lens assembly can be adjusted using a remote control unit. The remote control unit can be a wired or wireless remote control unit. The remote control unit can enable a user to increase or decrease the optical power provided by the fine tuning lens assembly. The remote control unit can communicate with the lens assembly based on the input it receives from the user. In turn, the optical power provided by the lens assembly and experienced by the patient can be modified.

As an example, the optical power change experienced by the user can be in discrete steps of approximately ⅛^{th} (0.125 D) of a Diopter. The optical power provided by the lens assembly can be adjusted as many times as necessary and for as long as necessary to determine a fine tuning vision correction for a patient. The “best” fine tuning setting of the lens assembly can be determined by the administrator or the patient or can at least be determined based on subjective patient feedback.

At step 208, a selected or final fine tuning correction provided by the lens assembly of the present invention can be displayed visually. As a fine tuning correction is being determined, and after a fine tuning correction setting of the lens assembly has been determined, a display device can visually indicate an optical power setting of the fine tuning lens assembly.

At the end of step 208, a patient's vision correction can be determined down to approximately ⅛^{th} of a Diopter. The patient's finely tuned vision correction can be added to the coarse vision correction determined at step 202 to determine the patient's total vision correction (i.e., combined coarse and finely tuned vision corrections).

A vision examination incorporating use of a fine tuning lens assembly of the present invention has numerous advantages over a conventional eye examination. During conventional eye examinations, patients often hesitate to request additional changes to the vision correction determined by the administrator of the eye examination. This can result in an inaccurate vision correction for the patient. By using the fine tuning lens assembly of the present invention, and by allowing the patient the ability to control a fine tuning optical power adjustment, the patient is more likely to feel comfortable toggling through the settings many times until satisfied that the best acuity has been achieved. In turn, the administrator can determine a more accurate or more precise vision prescription. Consequently, the eye examination is likely to end with the patient feeling that they are more involved in the final prescription decision which can result in less confusion, remorse or dissatisfaction with the determined prescription.

Additionally, a fine tuning lens assembly of the present invention enables the final more precise prescription to be determined with the use of electro-active lenses that provide optical powers that change almost instantaneously (i.e., on the order of milliseconds). With a conventional phoropter, a lens drum must be rotated from one position to another in order to change the optical power correction experienced by a patient. This requirement can take additional time when toggling between the two power settings. As a result, the patient can experience a brief moment of blackness as the lenses are changed. This moment of vision loss can be a disturbance and can make it more difficult for the patient to compare the relative acuity of the two power positions. With the electro-active lens or lenses in the fine tuning assembly, there is no blackout as the power changes quickly from one setting to another, thereby reducing the disturbances experienced by the patient.

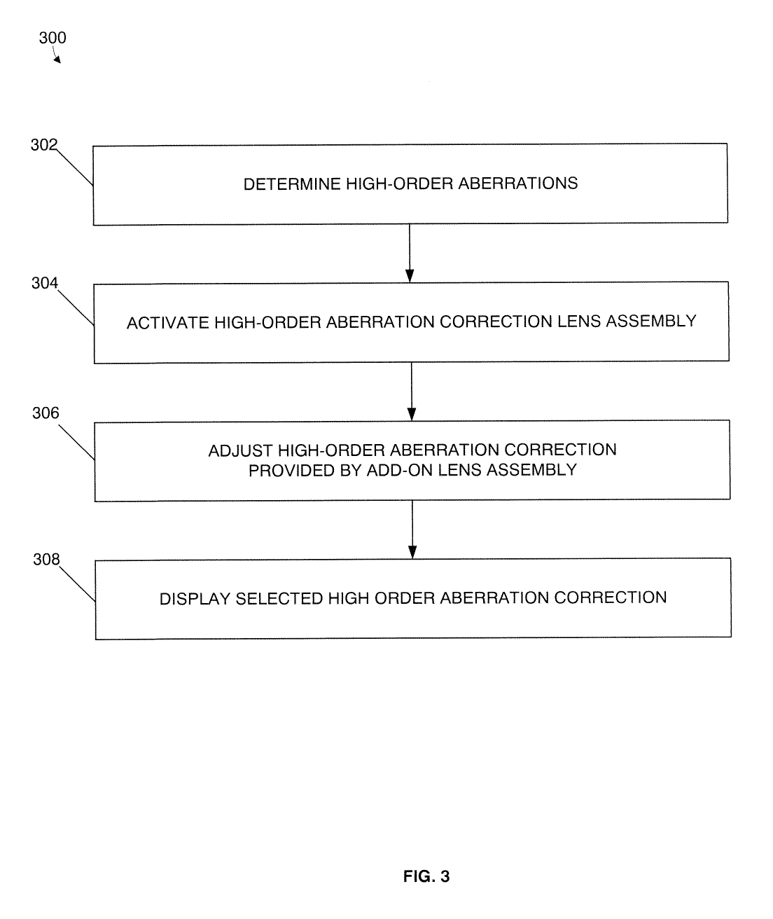

FIG. 3 provides a flowchart 300 that illustrates operational steps for providing a high-order aberration correction in accordance with an aspect of the present invention. The invention is not limited to this operational description. Rather, it will be apparent to persons skilled in the relevant art(s) from the teachings herein that other operational control flows are within the scope and spirit of the present invention. In the following discussion, the steps in FIG. 3 are described.

At step 302, high-order aberrations of patient's eye can be determined.

The high-order aberrations of the patient can be determined, for example, using a wavefront analyzer.

At step 304, a lens assembly providing high-order aberration correction capability of the present invention can be activated. The lens assembly can be an add-on lens assembly (such as the optical device 100 described above) or can be a lens assembly incorporated into the design and fabrication of the conventional phoropter. The lens assembly can include one or more electro-active lenses, one or more conventional lenses, or any combination thereof. The lens assembly can be placed within the optical path of the patient's eye.

At step 306, the high-order aberration correction capability of the lens assembly can be adjusted or tuned. The tuning of the lens assembly can be by the administrator of the eye examination. The lens assembly can be adjusted using a wired or wireless remote control. According to an aspect of the present invention, after high-order aberration information for a patient is collected, the information can be provided to the lens assembly of the present invention. The lens assembly can then provide a self-adjusted or automatic correction based on the received data to cancel out or correct for the provided and previously measured high-order aberrations.

At step 308, the determined high-order aberration correction can be displayed visually. As a high-order aberration correction is being determined, and after a high-order aberration correction setting of the lens assembly has been determined, a display device can visually indicate a high-order aberration correction setting of the lens assembly of the present invention.

At the end of step 308, a high-order aberration correction is provided to a patient by the lens assembly. As such, the patient can experience vision with high-order aberration correction. The patient, as a result, can be provided with better visual acuity than could be achieved with a standard or conventional eye examination.

An examination that provides a patient with the ability to visually experience high-order aberration correction can be a pre-cursor to refractive surgery on the patient intended to correct for high-order aberrations. The use of these adaptive lenses would allow the patient to experience the outcome of the customized refractive surgery before undergoing the procedure. Another purpose would be to provide a prescription for vision correction devices, such as, but not limited to, spectacle lenses, contact lenses, intra-ocular lenses, and corneal inlays that would correct all or some of the high-order aberrations in a patient's eye.

According to an aspect of the present invention, a phoropter of the present invention can include one or more electro-active lenses, one or fluid (or fluidic) or liquid lenses, one or more conventional lenses, or any combination thereof. Such a phoropter of the present invention can comprise a first set of lenses to provide coarse vision correction, a second set of lenses to provide fine vision correction, and a third set of lenses to provide high-order aberration correction. The lenses in each set can comprise as few as a single lens and/or can comprise any combination of conventional, fluid, or electro-active lenses.

As an example, a phoropter of the present invention can include a single electro-active lens, one or more fluid or liquid lenses and one or more conventional lenses. Each of the lenses of the phoropter can be in optical communication with one another. The one or more fluid or liquid lenses in combination with the one or more conventional lenses can provide a wide range of optical powers to provide coarse vision correction tuning capability (e.g., an optical power range of −15.0 D to 15.0 D). The electro-active lens of the phoropter can provide fine vision correction tuning capability. For example, the single electro-active lens can provide five optical power settings such as +0.25 D, +0.125 D, 0.0 D, −0.125 D, and −0.25 D. A second electro-active lens can be provided to enable the correction of high-order aberrations.

### Conclusion

While various embodiments of the present invention have been described above, it should be understood that they have been presented by way of example and not limitation. It will be apparent to one skilled in the pertinent art that various changes in form and detail can be made therein without departing from the spirit and scope of the invention. Therefore, the present invention should only be defined in accordance with the following claims and their equivalents.

What is claimed is:

1. An optical device, comprising:
one or more electro-active lenses;
wherein:
the optical device is attached to a phoropter and provides a fine vision correction;
the phoropter provides a coarse vision correction; and,
the fine vision correction and the coarse vision correction are optically combined.

2. The optical device of claim 1, wherein the optical device is attached mechanically to the phoropter.

3. The optical device of claim 1, wherein the optical device is attached magnetically to the phoropter.

4. The optical device of claim 1, wherein the optical device is attached adhesively to the phoropter.

5. The optical device of claim 1, wherein the optical device is positioned over an eye-well of the phoropter such that lenses of the phoropter are in optical communication with the one or more electro-active lenses of the optical device.

6. The optical device of claim 5, wherein the coarse vision correction provided by the lenses of the phoropter is adjusted by the fine vision correction provided by the one or more electro-active lenses of the optical device.

7. The optical device of claim 1, wherein the one or more electro-active lenses each provide a variable optical power comprising a negative, neutral or positive optical power.

8. The optical device of claim 7, wherein the one or more electro-active lenses are each continuously tunable over a range of optical powers.

9. The optical device of claim 7, wherein the one or more electro-active lenses are discretely tunable over a range of optical powers.

10. The optical device of claim 9, wherein the one or more electro-active lenses are discretely tunable in steps of approximately 0.125 D.

11. The optical device of claim 10, wherein the one or more electro-active lenses provide optical powers of +0.25 D, +0.125 D, 0.0 D, −0.125 D, and −0.25 D.

12. The optical device of claim 9, wherein the one or more electro-active lenses are discretely tunable in steps of 0.25 D.

13. The optical device of claim 12, wherein the one or more electro-active lenses provide optical powers of +0.5 D, +0.25 D, 0.0 D, −0.25 D and −0.5 D.

14. The optical device of claim 1, further comprising a remote control unit adapted to adjust the fine vision correction provided by the one or more electro-active lenses of the optical device.

15. The optical device of claim 14, wherein the remote control unit is wirelessly coupled to the optical device.

16. The optical device of claim 1, wherein the one or more electro-active lenses provide spherical correction.

17. The optical device of claim 1, wherein the one or more electro-active lenses provide cylindrical correction.

18. The optical device of claim 1, wherein the one or more electro-active lenses provide a high-order aberration correction.

19. A method for determining a vision correction for a patient, comprising:
determining a coarse vision correction for the patient with lenses of a phoropter;
determining a fine vision correction for the patient with a lens assembly coupled to the phoropter, the lens assembly comprising one or more electro-active lenses; and
combining the coarse vision correction and the fine vision correction to determine the vision correction for the patient.

20. A method for providing a high-order aberration correction to a patient, comprising:
determining high-order aberration information for the patient with a wavefront analyzer;
providing the high-order aberration information to a lens assembly coupled to a phoropter, the lens assembly comprising one or more electro-active lenses; and
adjusting the one or more electro-active lenses of the lens assembly to provide the high-order aberration correction to the patient.
